(19) United States
(12) Patent Application Publication
(10) Pub. No.: US 2022/0230718 A1
(43) Pub. Date: Jul. 21, 2022

(54) HEALTHCARE APPLICATION INSIGHT VELOCITY AID

(71) Applicant: International Business Machines Corporation, Armonk, NY (US)

(72) Inventors: Paul R. Bastide, Ashland, MA (US); Shakil Manzoor Khan, Highland Mills, NY (US); Senthil Bakthavachalam, Yorktown Heights, NY (US)

(21) Appl. No.: 17/154,200

(22) Filed: Jan. 21, 2021

(51) Int. Cl. G16H 10/65; G06F 16/245; G06F 16/23; G16H 20/10

(52) U.S. Cl. CPC G16H 10/65; G16H 20/10; G06F 16/23; G06F 16/245

(57) ABSTRACT

Aspects of the invention include receiving, by a processor, medical data associated with a patient, populating a patient ontology for the patient with the medical data, determining a completeness the patient ontology for the patient based at least in part on the medical data, querying an upstream data source based on the completeness of the patient ontology, updating the patient ontology based on a query response from the upstream data source, analyzing the updated patient ontology to determine an insight for the patient, and enacting an action based on the insight for the patient.

### BACKGROUND

The present invention generally relates to electronic medical records, and more specifically, to healthcare application insight velocity aids.

An Electronic Medical Record (EMR), or Electronic Health Record, is a digital record of a patient's medical history. An EMR tracks a patient's medical history over time and may include a range of data including both unstructured and structure data. Examples of unstructured data include notes by a variety of medical care providers, for example clinician notes. Examples of structured data include procedures performed, lab results, and medications taken.

A healthcare network typically comprises multiple source systems (e.g., a source of electronic medical records including electronic healthcare records (EHR), records from a claims system, lab feed, various data sources implementing the Health Level Seven (HL7) standard, patient satisfaction survey, etc.) and applies analytics to various electronic medical records (e.g., EHR, claims system, lab feed, HL7, patient satisfaction survey, etc.) to produce results for a desired population (e.g., patients, healthcare providers, insurance providers, provider organizations or networks, etc.). Communication between different components or systems in a healthcare network is typically implemented as an event driven processing system. Conventional event streaming systems primarily focus on single-server extract, transform and load (ETL) processing. Scalability is very limited for the conventional event streaming systems. In some cases, these systems can be scaled using traditional scaling techniques, such as load balancers and manually configured routing, to balance the transmission of stream data between nodes in the system. They also employ traditional resilience and replication patterns to the stream processing, including high availability proxy, persisting stored data to files and RDBMSs, and replicating between nodes based on manual configurations.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

Multi-tenant healthcare solutions attempt to accumulate electronic health records (EHR)/protected health information (PHI) and medical event data which co-exist from multiple vendors, customers, and ordinations in a single logical data processing system. Data can be added to these systems using an extraction-transformation-load (ETL) pipeline to load the data into a data lake, data reservoir, and data mart. As a new data element (HL7 message, admission, discharge, transfer (ADT) message, fast healthcare interoperability resources (FHIR) resource bundle) arrives, a pipeline executes stages to ETL each data into the system.

Each message requires many seconds to fully process through the ETL as the medical data has a high degree of outbound references (e.g., medication, medication orders, medical devices, observations, and medical events). As new messages are queued for processing, the ELT is forced to sequentially process the loading of the data processing system. As an intermediate step, the ELT spreads the load out across many worker threads, which execute the ETL logic. This ETL logic only scales so far for highly referential data elements as the data elements are loaded into the data processing systems. With the varied importance of real-time access of healthcare insights for each client, there is a need to optimize the access to the processed data so customers effectively derives patient and population insights.

### SUMMARY

Embodiments of the present invention are directed to a computer-implemented method for healthcare velocity insights. A non-limiting example of the computer-implemented method includes receiving, by a processor, medical data associated with a patient, populating a patient ontology for the patient with the medical data, determining a completeness the patient ontology for the patient based at least in part on the medical data, querying an upstream data source based on the completeness of the patient ontology, updating the patient ontology based on a query response from the upstream data source, analyzing the updated patient ontology to determine an insight for the patient, and enacting an action based on the insight for the patient.

Embodiments of the present invention are directed to a system for healthcare velocity insights. A non-limiting example of the system includes a processor communicative coupled to a memory, the processor operable to receiving, by a processor, medical data associated with a patient, populating a patient ontology for the patient with the medical data, determining a completeness the patient ontology for the patient based at least in part on the medical data, querying an upstream data source based on the completeness of the patient ontology, updating the patient ontology based on a query response from the upstream data source, analyzing the updated patient ontology to determine an insight for the patient, and enacting an action based on the insight for the patient.

Embodiments of the invention are directed to a computer program product for healthcare velocity insights, the computer program product comprising a computer readable storage medium having program instructions embodied therewith. The program instructions are executable by a processor to cause the processor to perform a method. A non-limiting example of the method includes receiving, by a processor, medical data associated with a patient, populating a patient ontology for the patient with the medical data, determining a completeness the patient ontology for the patient based at least in part on the medical data, querying an upstream data source based on the completeness of the patient ontology, updating the patient ontology based on a query response from the upstream data source, analyzing the updated patient ontology to determine an insight for the patient, and enacting an action based on the insight for the patient.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a cloud computing environment according to one or more embodiments of the present invention;

FIG. 2 depicts abstraction model layers according to one or more embodiments of the present invention;

FIG. 3 depicts a block diagram of a computer system for use in implementing one or more embodiments of the present invention;

FIG. 4 depicts a system for aiding healthcare insights according to embodiments of the invention;

FIG. 5 depicts a diagram of an exemplary graph of a patient ontology according to one or more embodiments of the invention; and

FIG. 6 depicts a flow diagram of a method for healthcare velocity insights according to one or more embodiments of the invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

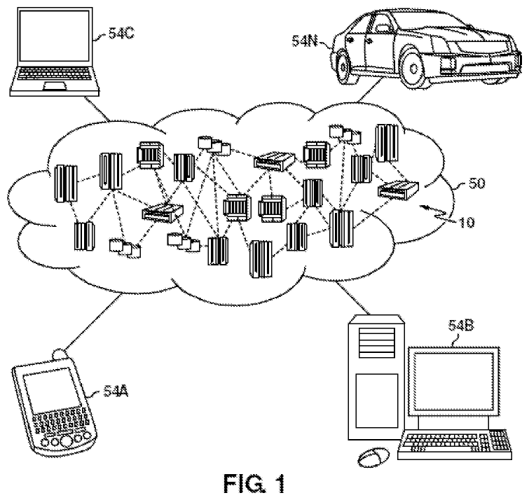

Referring now to FIG. 1, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

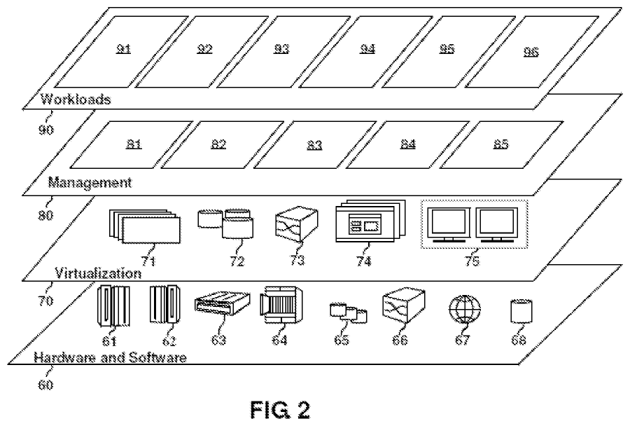

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and healthcare insights velocity aid 96.

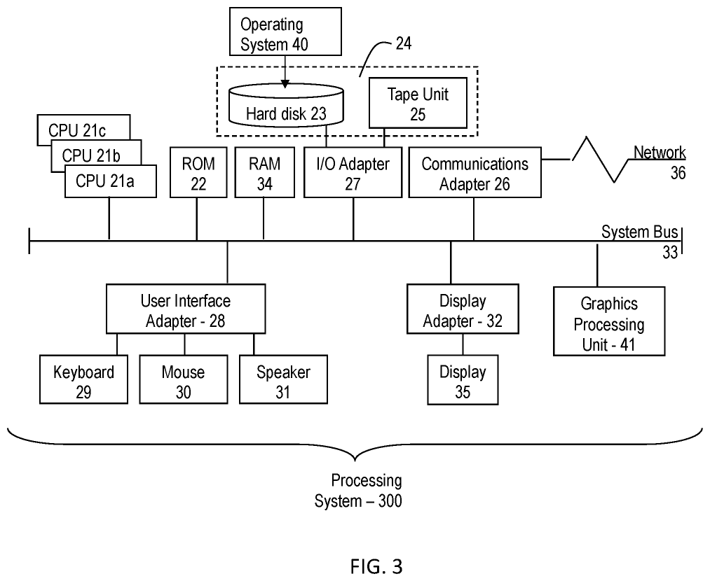

Referring to FIG. 3, there is shown an embodiment of a processing system 300 for implementing the teachings herein. In this embodiment, the system 300 has one or more central processing units (processors) 21a, 21b, 21c, etc. (collectively or generically referred to as processor(s) 21). In one or more embodiments, each processor 21 may include a reduced instruction set computer (RISC) microprocessor. Processors 21 are coupled to system memory 34 and various other components via a system bus 33. Read only memory (ROM) 22 is coupled to the system bus 33 and may include a basic input/output system (BIOS), which controls certain basic functions of system 300.

FIG. 3 further depicts an input/output (I/O) adapter 27 and a network adapter 26 coupled to the system bus 33. I/O adapter 27 may be a small computer system interface (SCSI) adapter that communicates with a hard disk 23 and/or tape storage drive 25 or any other similar component. I/O adapter 27, hard disk 23, and tape storage device 25 are collectively referred to herein as mass storage 24. Operating system 40 for execution on the processing system 300 may be stored in mass storage 24. A network adapter 26 interconnects bus 33 with an outside network 36 enabling data processing system 300 to communicate with other such systems. A screen (e.g., a display monitor) 35 is connected to system bus 33 by display adaptor 32, which may include a graphics adapter to improve the performance of graphics intensive applications and a video controller. In one embodiment, adapters 27, 26, and 32 may be connected to one or more I/O busses that are connected to system bus 33 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 33 via user interface adapter 28 and display adapter 32. A keyboard 29, mouse 30, and speaker 31 all interconnected to bus 33 via user interface adapter 28, which may include, for example, a Super I/O chip integrating multiple device adapters into a single integrated circuit.

In exemplary embodiments, the processing system 300 includes a graphics processing unit 41. Graphics processing unit 41 is a specialized electronic circuit designed to manipulate and alter memory to accelerate the creation of images in a frame buffer intended for output to a display. In general, graphics processing unit 41 is very efficient at manipulating computer graphics and image processing and has a highly parallel structure that makes it more effective than general-purpose CPUs for algorithms where processing of large blocks of data is done in parallel.

Thus, as configured in FIG. 3, the system 300 includes processing capability in the form of processors 21, storage capability including system memory 34 and mass storage 24, input means such as keyboard 29 and mouse 30, and output capability including speaker 31 and display 35. In one embodiment, a portion of system memory 34 and mass storage 24 collectively store an operating system coordinate the functions of the various components shown in FIG. 3.

Turning now to an overview of technologies that are more specifically relevant to aspects of the invention, in big data applications such as a multi-tenant healthcare system, data generally flows from various data sources (also called data streams) into a data reservoir where the data is eventually processed and stored in a data warehouse and/or data mart for consumption by various applications, such as business intelligence tools.

Data reservoirs enable all forms of customer (e.g., healthcare providers) specific data to be stored in a uniform, single large storage repository for access by a data processing engine. Data reservoirs may be used for multi-dimensional analytics to discover optimal business outcomes. Data reservoirs may be single-tenant, where the data is stored and owned by a single entity, or multi-tenant, where data is stored and owned by multiple entities. Multi-tenant data reservoirs isolate specific tenant data from all other tenants. Multi-tenant data reservoirs may maximize storage use of a database and provide uniform security and decryption of data. The data reservoir may include certain predetermined permissions, such as, for example, read-only access to one or more preselected systems and read/write access to other preselected systems.

Similarly, a data warehouse is a central repository of integrated data from one or more disparate data sources. They store current and historical data in one single place that are used for creating analytical reports for workers throughout the enterprise. The data warehouse may include certain predetermined permissions, such as, for example, read-only access to one or more preselected systems and read/write access to other preselected systems.

Various cloud-based health record systems providers offer multi-tenant healthcare solutions where Electronic Health Records (“EHR”), Protected Healthcare Information (“PHI”), and/or patient medical data are stored together from multiple vendors, customers, and/or organizations in a single database and/or logical processing engine. Exemplary vendors may include, for example, hospitals, insurance providers, pharmacies, health care providers, etc. Data elements (which may be, e.g., structured and/or unstructured data) from various sources may be processed using an Extraction-Transformation-Load (“ETL”) system to thereby load the data into the data reservoir and/or a data mart for consumption by a specific business group. As a new data element (e.g., HL7 message, ADT message) is received, a pipeline may execute stages to complete the ETL process.

ETL is normally a continuous, ongoing process with a well-defined workflow. ETL first extracts data from structured or unstructured data sources. Then, data is cleansed, enriched, transformed, and stored either back in the data reservoir or in a data warehouse (or data mart within the data warehouse). Each incoming message (1 Kilobyte, 1 Gigabyte) may require a period of time (e.g., several seconds) to fully process through the ETL system. As new messages are queued for processing by the ETL system, ETL systems generally sequentially process the new messages thereby uploading the processed data/message to a data mart. As an intermediate step, the ETL system may spread the load out across many systems, which execute the ETL.

Turning now to an overview of the aspects of the invention, one or more embodiments of the invention provide for systems and processes for aiding healthcare insight velocity. Healthcare insights refers to a patient specific or population specific that allows for an intervention to enhance and/or improve a patient or patient population's general well-being. These insights are derived from the available data utilizing the techniques described above from disparate sources of medical information for the patient and/or patient population. Patient population refers to patients that are similarly situated due to demographics, medical conditions, and the like.

For developing healthcare insights, completeness of the patient data for each patient is an essential feature. Incomplete or stale data for a patient can cause a multitude of issues including incorrect medication dosages for patients with chronic conditions. One or more embodiments of the present invention, systems described herein can aide in generating a patient ontology having a high degree of completeness. The system can access patient data using a variety of techniques such as ELT or FHIR bundles of data requests. To calculate a completeness of this patient data, the system can use a FHIR v. 3 ontology as a standard and populate the ontology with the data received from the FHIR bundle. Based on the determined completeness, the system attempts to supplement missing or stale data in the patient ontology by generate upstream interrogations to a variety of applications in a multi-tenant healthcare system and to the patient themselves. The upstream interrogations can include a survey sent to a medical device, an application in a healthcare system, and the patient. The interrogations can also include a query to an EHR/EMR system and/or a data request for subsequent data deliveries. After the upstream interrogations are responded to and the requested data is received, the patient ontology can be augmented now to include both data from the initial FHIR bundle and the responses to the upstream interrogations.

In one or more embodiments of the invention, once the patient ontology has a high enough degree of completeness, the system can then generate interventions for the patient. Interventions can include a variety of actions including, but not limited to, notifying a healthcare provider, adjusting a prescription dosage, changing a medication type, and/or suggesting changes to the patient's lifestyle. The patient ontology can be updated periodically or continuously using upstream interrogatories for real-time, continuous monitoring of the patient to develop future healthcare insights for the patient. After the initial ontology is build, the system can monitor data as it become outdated or stale (e.g., 90+ days old, for example) and survey or interrogate a variety of sources to update this data. In addition, in one or more embodiments of the invention, the system receives continuous and/or intermittent dosage, treatment protocol, and medicine compliance data for a patient to track adherence to a treatment protocol for an acute or chronic medical condition. For example, a patient following a diabetic treatment protocol can have one or more wearable medical devices that measure blood glucose levels and delivers insulin to the patient. This data can be communicated to the system to update the patient ontology and provide for further healthcare insights for the patient.

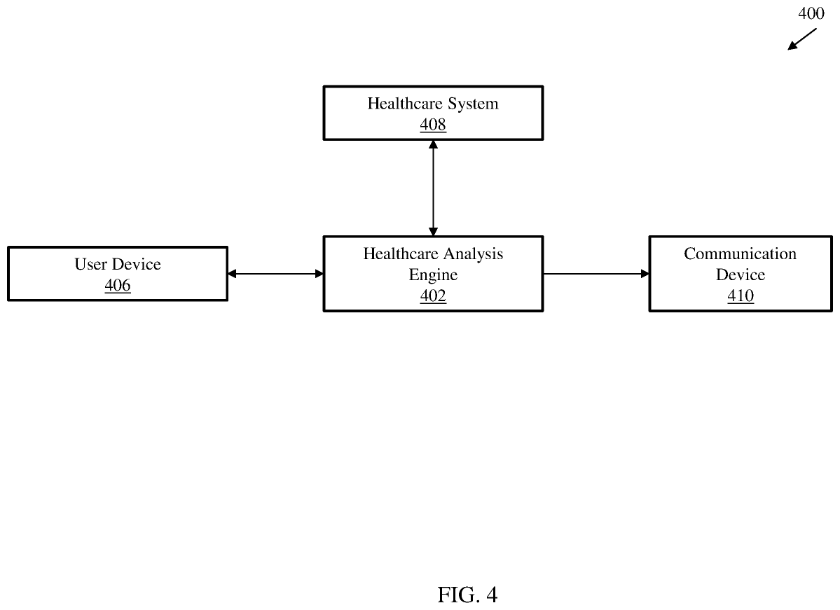

Turning now to a more detailed description of aspects of the present invention, FIG. 4 depicts a system for aiding healthcare insights according to embodiments of the invention. The system 400 includes a healthcare analysis engine 402, a user device 406, a communication device 410, and a healthcare system 408.

In one or more embodiments of the invention, the healthcare analysis engine 402 can be implemented on the processing system 300 found in FIG. 3. Additionally, the cloud computing system 50 can be in wired or wireless electronic communication with one or all of the elements of the system 400. Cloud 50 can supplement, support or replace some or all of the functionality of the elements of the system 400. Additionally, some or all of the functionality of the elements of system 400 can be implemented as a node 10 (shown in FIGS. 1 and 2) of cloud 50. Cloud computing node 10 is only one example of a suitable cloud computing node and is not intended to suggest any limitation as to the scope of use or functionality of embodiments of the invention described herein. The healthcare system 408 can include any single or multitenant healthcare platform containing EHR/PHI configured to send and receive data using ETL, HL7 messages, ADT messages, FHIR resource bundles, and the like. The user device 406 can be any type of device for a patient including a smart watch, smart phone, laptop, and the like. Further user device 406 can also include medical devices such as medication delivery devices, biometric monitoring devices, medication compliance devices, and the like.

In one or more embodiments of the invention, the healthcare analysis engine 402 can determine healthcare insights for a patient by building a patient ontology. A standard patient ontology can be utilized such as a FHIR DSTU3 (draft standard for trial use version 3) ontology. The healthcare analysis engine 402 can be installed, for example, on the user device 406 to aid in tracking compliance with a treatment protocol or the healthcare analysis engine 402 can be installed on a remote server that can be accessed through the user device 406 or other communication means. The user device 406 is utilized to communicate data associated with the treatment protocol of the patient with the healthcare analysis engine 402. The treatment protocol data can include when a patient takes a prescribed medication, how much of the medication is taken (dosage), any biometric data taken by the user device 406 or entered in by the patient such as, for example, blood pressure readings, blood glucose data, heart rate, and the like. This treatment protocol data can be transmitted to the healthcare analysis engine 402 which can push this data to the healthcare system 408 using an FHIR bundle (observation (data) with a patient identifier). The healthcare analysis engine 402 can calculate the completeness of the patient ontology and determined missing or stale data. For example, if a patient ontology is missing data or has stale data for a height and weight. The healthcare analysis engine 402 can send upstream interrogations to both the user device 406 as a survey to the patient and/or to the healthcare system 408 to query this data. The interrogations to the upstream healthcare system 408 can be in the form of FHIR syntax as follows: SELECT*FROM FHIR.WEIGHT WHERE PTNT_IDENTIFIER=‘USER A’; SELECT*FROM FHIR.WEIGHT WHERE PTNT_IDENTIFIER=‘USER A’; and SELECT*FROM FHIR.PROFILE WHERE PTNT_IDENTIFIER=‘USER A’.

In one or more embodiments of the invention, the upstream interrogations can first attempt to secure the data from the healthcare system 408. If this data does not exist within the healthcare system 408, the healthcare analysis engine 402 can forward a survey/notification to the patient's user device 406 with questions such as: What is your height? What is your current weight?, etc. In addition, the healthcare analysis engine 402 can query the user device 406 directly to access biometric information associated with the patient to update the patient ontology. While this information is considered protected health information (PHI), the communication means and other techniques for securing this information can take into account the sensitivity and security of the data being requested and utilize commercially reasonable safeguards such as encryption, de-identification, and the like in compliance of relevant rules and regulations protecting such information.

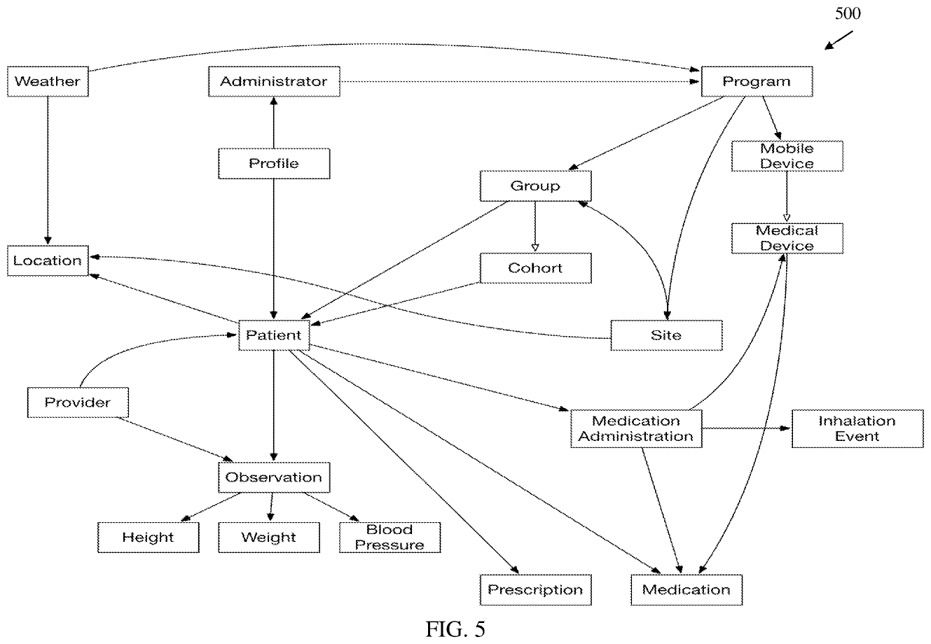

In one or more embodiments of the invention, the healthcare analysis engine 402 can calculate the completeness of the patient ontology by generating of a graph of the ontology and loading in the received FHIR bundle data into the graph. FIG. 5 depicts a diagram of an exemplary graph of a patient ontology according to one or more embodiments of the invention. The graph 500 includes a plurality of nodes and edges that symbolize connections between resources in the patient ontology. The healthcare analysis engine 402 can calculate the completeness of the graph 500 by associating a lease set of connections between the resources based on the patient identifiers. Further, the healthcare analysis engine 402 uses a variety of techniques such as calculating the connectivity count, total edges, total unique resources, and/or any other graph analysis techniques (e.g., subset coverage). The healthcare analysis engine 402 can establish a minimum threshold of completeness before sending upstream interrogations such a survey to the patient or when a necessary edge in the graph does not exist for a resource. In one or more embodiments, the healthcare analysis engine 402 can determine completeness based on needed data surrogate key lookups.

In one or more embodiments of the invention, the healthcare analysis engine 402 can communicate with the user device 406 or through the communication device 410 to enact an action once the patient ontology is complete or has a threshold level of completeness. The action can be based on the real-time and/or near real-time data collected from the user device 406 that can include data about a treatment protocol and/or medication compliance. The action can include notifying a healthcare professional associated with a patient through the communication device 410. The notification can be an email or other type of electronic message to the healthcare provider and can be structured using standard such as HL7 messages and/or FHIR bundles. The action can also include sending instructions to the user device to adjust a medication dosage. For example, if the user device is an insulin pump, the action could be instructions to change the dosage based on the data collected and the updated patient ontology. Further, the action can include a change in a prescription for the patient sent to the patient, pharmacist, or both.

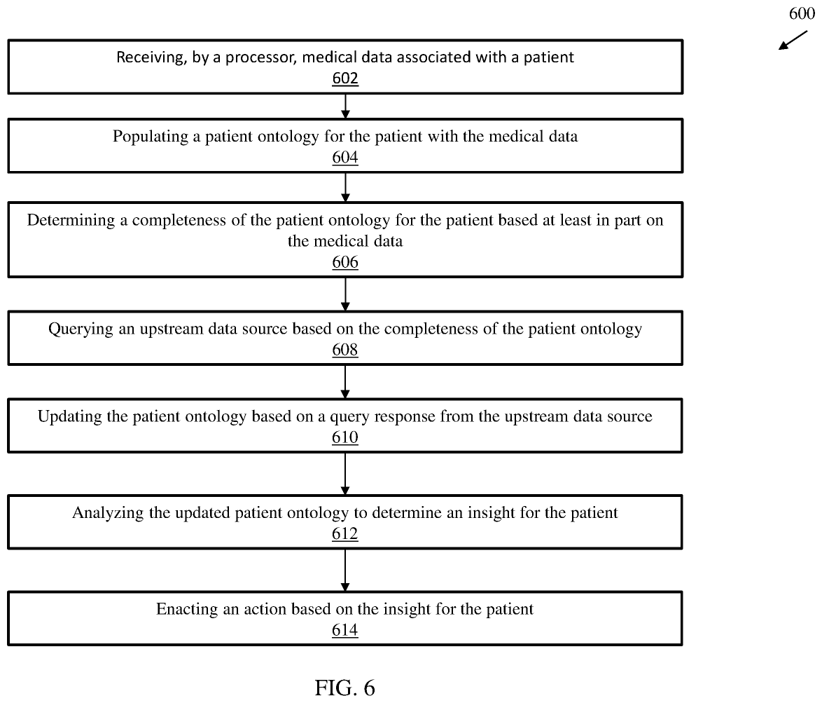

FIG. 6 depicts a flow diagram of a method for healthcare velocity insights according to one or more embodiments of the invention. The method 600 includes receiving, by a processor, medical data associated with a patient, as shown in Block 602. At block 604, the method 600 includes populating a patient ontology for the patient with the medical data. The patient ontology can be taken from a model ontology and be a representation of the medical history and current medical insights for the patient. The method 600 continues at block 606 by including determining a completeness of the patient ontology for the patient based at least in part on the medical data. Also, the method 600 includes querying an upstream data source based on the completeness of the patient ontology, as shown at block 608. The method 600 then includes updating the patient ontology based on a query response from the upstream data source, as shown in block 610. Also, the method 600 includes analyzing the updated patient ontology to determine an insight for the patient. And at block 612, the method 600 includes enacting an action based on the insight for the patient.

Additional processes may also be included. It should be understood that the processes depicted in FIG. 6 represent illustrations, and that other processes may be added or existing processes may be removed, modified, or rearranged without departing from the scope and spirit of the present disclosure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
receiving, by a processor, medical data associated with a patient;
populating a patient ontology for the patient with the medical data;
determining a completeness of the patient ontology for the patient based at least in part on the medical data;
querying an upstream data source based on the completeness of the patient ontology;
updating the patient ontology based on a query response from the upstream data source;
analyzing the updated patient ontology to determine an insight for the patient; and
enacting an action based on the insight for the patient.

2. The computer-implemented method of claim 1, wherein the action comprises adjusting a prescription for the patient.

3. The computer-implemented method of claim 1, wherein the action comprises sending a notification to a healthcare provider for the patient.

4. The computer-implemented method of claim 1, wherein the action comprises transmitting medication dosage instructions for medical device.

5. The computer-implemented method of claim 4, wherein the medication dosage instructions adjust dispensing of a medication dosage by a medical device associated with the patient.

6. The computer-implemented method of claim 5, wherein the medical device comprises a wearable device for the patient.

7. The computer-implemented method of claim 1, wherein determining the completeness of the patient ontology comprises analyzing the patient ontology to determine at least one of:
one or more stale data points; and
one or more incomplete data points.

8. The computer-implemented method of claim 7, further comprising: querying the patient for data associated with at least one of the one or more stale data points and the one or more incomplete data points.

9. The computer-implemented method of claim 7, wherein the one or more stale data points comprise data points that are older than a threshold time period.

10. The computer-implemented method of claim 8, wherein querying the patient comprises: accessing a wearable device for the patient to obtain updated biometric data for the patient.

11. A system comprising:
a processor communicatively coupled to a memory, the processor configured to:
receive medical data associated with a patient;
populate a patient ontology for the patient with the medical data;
determine a completeness of the patient ontology for the patient based at least in part on the medical data;
query an upstream data source based on the completeness of the patient ontology;
update the patient ontology based on a query response from the upstream data source;
analyze the updated patient ontology to determine an insight for the patient; and
enact an action based on the insight for the patient.

12. The system of claim 11, wherein the action comprises adjusting a prescription for the patient.

13. The system of claim 11, wherein the action comprises sending a notification to a healthcare provider for the patient.

14. The system of claim 11, wherein the action comprises transmitting medication dosage instructions for medical device.

15. The system of claim 14, wherein the medication dosage instructions adjust dispensing of a medication dosage by a medical device associated with the patient.

16. A computer program product for healthcare insights comprising a computer readable storage medium having program instructions embodied therewith, the program instructions executable by a processor to cause the processor to perform a method comprising:
receiving, by a processor, medical data associated with a patient;
populating a patient ontology for the patient with the medical data;
determining a completeness of the patient ontology for the patient based at least in part on the medical data;
querying an upstream data source based on the completeness of the patient ontology;
updating the patient ontology based on a query response from the upstream data source;
analyzing the updated patient ontology to determine an insight for the patient; and
enacting an action based on the insight for the patient.

17. The computer program product of claim 16, wherein the action comprises adjusting a prescription for the patient.

18. The computer program product of claim 16, wherein the action comprises sending a notification to a healthcare provider for the patient.

19. The computer program product of claim 16, wherein the action comprises transmitting medication dosage instructions for medical device.

20. The computer program product of claim 19, wherein the medication dosage instructions adjust dispensing of a medication dosage by a medical device associated with the patient.
